(19) United States
(12) Patent Application Publication
(10) Pub. No.: US 2025/0032276 A1
(43) Pub. Date: Jan. 30, 2025

(54) PROSTHETIC TRAINING DEVICE

(71) Applicant: Sheffield Hallam University, Sheffield South Yorkshire (GB)

(72) Inventors: Ivan PHELAN, Sheffield South Yorkshire (GB); Nicholas DULAKE, Sheffield South Yorkshire (GB)

(73) Assignee: Sheffield Hallam University, Sheffield South Yorkshire (GB)

(21) Appl. No.: 18/714,212

(22) PCT Filed: Nov. 28, 2022
(86) PCT No.: PCT/GB2022/053009

§ 371 Date: May 29, 2024

(30) Foreign Application Priority Data
Nov. 29, 2021 (GB) 2117236.6

(51) Int. Cl. A61F 2/50; A63B 21/00; A63B 22/00; A63B 23/035

(52) U.S. Cl. CPC A61F 2/50; A63B 21/4001; A63B 23/03508; A63B 2022/0092

(57) ABSTRACT

The invention relates to prosthetic training devices for attachment to the residual limb of transradial or transhumeral amputee.

### FIELD OF THE INVENTION

The invention relates to prosthetic training devices for attachment to the residual limb of transradial or transhumeral amputee. More specifically, the present invention is concerned with a device for use with immersive virtual reality (IVR) training systems to assist upper limb amputees in developing the necessary skills to effectively operate a myoelectric prosthetic arm.

### BACKGROUND OF THE INVENTION

There are around 45,000 amputee and limb deficient people in England alone, where each year about 4,000 major lower limb amputees, 200 upper limb amputees and 150 congenital amputees are referred to specialist centres. Despite the lower incidence of upper limb amputations, these individuals' longer life expectancy means that they require rehabilitation support for more years than lower limb amputees. There are approximately 11,000 upper limb amputees in England requiring maintenance of their prostheses and follow up.

Upper limb amputation is particularly challenging as, in addition to functional movements necessary for professional and social activity, the hand is critical for psychosocial acts including gestures, communication, and sensation. Thus the resultant changes in motor and sensory functions have a deleterious impact on upper limb amputees' activities of daily living (ADL). To minimize this impact, prosthetics centres provide training to amputees to make the best use of their prosthetics as an integral part of their overall management. In particular, myoelectric (Myo) prosthetics require intensive training in motor learning and cortical remapping, with the user needing to learn to consciously control muscle contraction, level of activation, and isolation through repetitive exercises, as well as getting used to the significant weight of the Myo prosthetic device.

For example, there are 35 NHS prosthetic centres in the UK which provide support to an average of nine new Myo prosthetic users annually, each of which undergoes a one-to-one intensive training programme. This initial training program includes at least six sessions in clinic, requiring travel for amputees which has been identified as negatively impacting their training. In addition, there are approximately 200 amputee-clinic teams in operation throughout the United States in rehabilitation centres, including military veterans' centres, which provide similar training to new Myo prosthetic users. However, training systems such as, e.g. Ottobock MyoBoy and Ossur VirtuLimb, which are typically used during initial Myo prosthetic training, are non-immersive in nature, expensive and are of limited functionality. Moreover, studies have suggested that limited pre-prosthetic training, both in terms of quality and duration (the UK NHS provides, on average, six clinic-based training sessions to new users), is closely associated with abandonment of expensive Myo prosthetic devices, with an estimated rejection rate of 35% in children and 23% in adults (NHS FIGS. 8%-50%). Other contributing factors resulting in low confidence in using and/or early abandonment of Myo prosthetic devices include the limited availability of occupational therapists, and the inability to provide access to training outside a clinic (e.g. within the home) environment.

There is, therefore, a clinical need to provide new prosthetic training devices for use with immersive virtual reality (IVR) Myo training systems, to assist upper limb amputees in developing the necessary skills to effectively operate a Myo prosthetic arm (single or multi-grip). In particular, the devices should be suitable for use with “off the shelf” virtual reality (VR) headsets and motion tracking controllers, and must correctly align such controllers at the correct place on the amputee's arm, enabling signals to be detected by the VR system during training. Furthermore, the device should be capable of mimicking the weight of a real Myo prosthetic arm, and must securely attach to the residual limb in order to establish user confidence during VR training.

The use of such training devices in an IVR Myo prosthetic training system would provide numerous benefits for amputees, occupational therapists and for health care providers:

For amputees, it could help improve the current training of amputees prior to committing to the use of a Myo prosthetic arm (as an upgrade from a body powered prosthetic arm). It could also be used as a further training aid to assist the development of advanced skills once a Myo prosthetic has been prescribed. Furthermore, the ability for patients to use the system, which mirrors the weight and feel of the final prosthetic arm, away from the clinical environment would reduce the pressure of intensive training, the strict schedule of clinic sessions and the travel, and so would lead to a reduction in the early abandonment rate of expensive (£20,000/device) Myo prosthetics.

For occupational therapists, such a Myo prosthetics training system could reduce workload by reducing the number of clinic visits a new prosthetic user would require. For healthcare providers such as the UK NHS, improved and partial home training should reduce the costs of clinic-based occupational therapist led training sessions and patients transport, and it could potentially reduce the rate of abandoned Myo prosthesis (approximately £20,000/device).

It is an aim of the present invention to provide such prosthetic training devices for use with IVR Myo training systems to assist upper limb amputees in developing the necessary skills to effectively operate a Myo prosthetic arm.

### STATEMENTS OF INVENTION

According to a first aspect of the invention there is provided a prosthetic training device for attachment to a residual limb of a transradial amputee, said device comprising:
(a) at least one elongate rigid support shaft;
(b) a connector configured to removably receive one or more weights and/or a virtual reality (VR) controller or other positional sensor, wherein said connector is engaged with and secured or securable in a fixed position along the distal part or half of said support shaft(s);
(c) a first securing strap comprising a first end and a second end, wherein said strap is engaged with and secured or securable in a fixed position along the proximal part or half of at least one of said support shaft(s), and wherein said strap is configured to selectively secure the first end to the second end around the residual limb of a user at a circumferential position below the elbow;
(d) a second securing strap comprising a first end and second end, wherein said strap is flexibly tethered or tetherable to said first securing strap and/or the proximal part or half of at least one of said support shaft(s), and wherein said strap is configured to selectively secure the first end to the second end around the residual limb of a user at a circumferential position above the elbow; and
(e) a forearm retainer configured to maintain a volumetric fit over the residual limb of a user via adjustable circumferential force, wherein said retainer is engaged with and secured or securable in a fixed position on at least one of said support shaft(s) between said connector and said first securing strap.

The terms ‘proximal’ and ‘distal’ as used herein are defined with respect to the centre of the body of the user when the device is in normal use. Therefore, as will be readily appreciated, the ‘distal’ part or half of an elongate shaft refers specifically to the part or half that is nearer to the shoulder of the user when the device is in normal use. Conversely, the ‘proximal’ part or half of an elongate shaft refers specifically to the part or half that is furthest from the shoulder of the user when the device is in normal use.

Advantageously, the provision of the connector within such a device allows for the correct positioning and alignment of a VR motion tracking controller or other positional sensor, preferably a VR motion tracking controller, at the correct special position on the amputee's (virtual) arm, enabling positional reference signals to be detected by the VR system during training. Furthermore, as the connector is configured to receive at least one, and preferably a plurality of weights, the device weight can be modified to accurately mimic the weight and dynamics of the final Myo prosthetic device for which the user is training to use.

In addition, the combination of two securing straps, tethered together wherein one strap is disposed above and one strap disposed below the elbow has been shown to provide a comfortable yet secure fit around the user's elbow.

Similarly the forearm retainer, provides a comfortable yet secure fit around the residual limb or the user. Moreover, by securing the device to the residual limb via an adjustable circumferential force (e.g. by inflation of an armband), circulation problems within the residual limb can be avoided. Preferably, the connector is slidably engaged with and securable in a plurality of positions along the distal part or half of the support shaft or support shafts. Such a configuration allows for positional adjustment of the connector, and by extension the position of attached weights and/or a VR positional controller or other positional sensor, preferably a VR positional controller, in use, along the length of the support shaft or support shafts to adjust the centre of mass to more accurately mimic the feel of the final Myo prosthetic device and/or to provide an accurate VR positional reference.

Similarly, the first securing strap is preferably slidably engaged with and securable in a plurality of positions along the proximal part or half of the support shaft or at least one, and preferably all of, said support shafts. Likewise, the forearm retainer is preferably slidably engaged with and securable in a plurality of positions along the length of the support shaft or at least one, and preferably all of, said support shafts. Such arrangements allow for adjustment of the position of first securing strap and forearm retainer to ensure an optimum fit is achieved.

In such embodiments wherein the connector, first securing strap and/or forearm retainer are slidably engaged with the support shaft(s), the connector, securing strap and/or forearm retainer are preferably reversibly secured in position on the support shaft(s) by a releasable locking mechanism. Although not limited to any particular arrangement, such a releasable locking mechanism can be provided by the variable application of friction to the surface of the support shaft(s) upon rotation of an abutting cam connected to said connector, securing strap or forearm retainer.

In preferred embodiments, the support shaft(s) is/are formed from carbon fibre. The use of such a material results in a stiff but negligible mass support structure that minimises the contribution of this fixed component to the perceived weight and/or dynamic properties of the training device. In some embodiments, the device comprises a single support shaft. In other embodiments, more than one, e.g. two, three or four, such shafts may be provided. Multiple, radially aligned, support shafts may be provided in embodiments where increase device stiffness, e.g., where heavy weights are to be supported, is required.

In further preferred embodiments, a portion, but not all, of said first and/or second securing strap is connected to a rigid support layer, wherein said support layer is secured or securable in position on said support shaft or at least one, and preferably all of, said support shafts. This arrangement not only facilitates a secure fit around the elbow of the user but also simplifies the process of fitting the device to the user.

The component part referred to as a forearm retainer is preferably provided in the form an inflatable armband, which is optionally formed from a thermoplastic sheet material. An exemplary thermoplastic sheet material is a polyurethane (TPU) sheet material, which may be ultrasonically welded to form an airtight enclosure. Preferably, the forearm retainer is disposed within a rigid support structure, e.g. a tube, wherein the support structure is secured or securable in position on said support shaft or at least one, and preferably all of, said support shafts. However, as the skilled reader will readily appreciate, the forearm retainer may likewise be disposed in a non-rigid inelastic support structure, such as a flexible inelastic strap or mesh. Such a support structure advantageously anchors or secures the forearm retainer in position on said support shaft(s), restraining movement of the retainer relative to the support shaft.

The connector and/or, if present, said support structure and/or said support layer(s) are preferably formed from a thermoplastic polymer, and more preferably are formed from 3D printable thermoplastic polymer such as acrylonitrile butadiene styrene (ABS), polylactic acid (PLA), polycarbonate (PC), polyamide, high impact polystyrene (HIPS) or high-density polyethylene (HDPE). In particularly preferred embodiments, these components are formed from ABS.

In preferred embodiments, the connector comprises a plurality of radially aligned chambers, wherein each chamber comprises at least one wall and, optionally, a base to define an inner chamber surface that is configured to removably accept a corresponding weight. In a particularly preferred embodiment, the connector comprises 6 chambers, each configured to receive a 75 g weight

Preferably, each inner chamber surface includes or is formed from a magnet. Such a configuration allows for the secure yet removable attachment of metal weights, thereby allowing the device to replicate the different weights of specific Myo prosthetics.

In preferred embodiments, the second securing strap is tethered to said first securing strap and/or the proximal part or half of said support shaft or at least one, and preferably all of, said support shafts via variable length elastic chord. Whilst not limited to any particular arrangement for achieving such variation of chord length, this can be effectively provided by a cleat configured to securely hold the chord in place and prevent slipping. This arrangement is advantageous in that it allows for adjustment of the position of the first securing strap relative to the second securing strap, thereby optimising the fit around the elbow of the user.

According to a second aspect of the invention there is provided a prosthetic training device for attachment to a residual limb of a transhumeral amputee, said device comprising:
(a) at least one first elongate rigid support shaft;
(b) at least one second elongate rigid support shaft, wherein the distal end of each of said at least one second support shaft is hingedly connected to the proximal end of the corresponding first support shaft via an articulated and optionally lockable joint;
(c) a connector configured to removably receive one or more weights and/or a virtual reality (VR) controller or other positional sensor, wherein said connector is engaged with and secured or securable in a fixed position along the distal part or half of said first support shaft(s);
(d) a securing strap comprising a first end and a second end, wherein said strap is engaged with and secured or securable in a fixed position along the proximal part or half of at least one of said second support shaft(s), and wherein said strap is configured to selectively secure the first end to the second end around a circumferential position of the residual limb of a user;
(e) a body harness flexibly tethered or tetherable to said securing strap and/or the proximal part or half of at least one of said second support shaft(s) and configured such that, in use, said harness wraps around the shoulder of the residual limb and around the torso of a user; and
(f) an arm retainer configured to maintain a volumetric fit over a portion of the residual limb of a user via adjustable circumferential force, wherein said retainer is engaged with and secured or securable in a fixed position on at least one of said second support shaft(s) between said articulated joint and said securing strap.

As per the device of the first aspect, the provision of the connector within such a device allows for the correct positioning and alignment of a VR motion tracking controller or other positional sensor, preferably a VR motion tracking controller, at the correct special position on the amputee's (virtual) arm, enabling positional reference signals to be detected by the VR system during training. Furthermore, as the connector is configured to receive at least one, and preferably a plurality of weights, the device weight can be modified to accurately mimic the weight and dynamics of the final Myo prosthetic device for which the user is training to use.

In addition, the combination of a securing strap and body harness which are tethered together has been shown to provide a comfortable yet secure fit to the residual limb of a transhumeral amputee user.

Further, and similar to the forearm retainer of the first aspect, the arm retainer provides a comfortable yet secure fit around the residual limb or the user. Moreover, by securing the device to the residual limb via an adjustable circumferential force (e.g. by inflation of an armband), circulation problems within the residual limb can be avoided. Preferably, the connector is slidably engaged with and securable in a plurality of positions along the length of the first support shaft(s). Such a configuration allows for positional adjustment of the connector, and by extension the position of attached weights and/or a VR positional controller or other positional sensor, preferably a VR positional controller, in use, along the length of the first support shaft(s) to adjust the centre of mass to more accurately mimic the feel of the final Myo prosthetic device and/or to provide an accurate VR positional reference.

Similarly, the securing strap is preferably slidably engaged with and securable in a plurality of positions along the proximal part or half of said second support shaft or at least one, and preferably all of, said second support shafts. Likewise, the arm retainer is preferably slidably engaged with and securable in a plurality of positions along the length of said second support shaft or at least one, and preferably all of, said second support shafts. Such arrangements allow for adjustment of the position of first securing strap and forearm retainer to ensure an optimum fit is achieved.

In such embodiments wherein the connector, securing strap and/or arm retainer are slidably engaged with the first or second support shaft(s), the connector, securing strap and/or arm retainer are preferably reversibly secured in position on said first or said second support shaft(s) by a releasable locking mechanism. Although not limited to such an arrangement, a releasable locking mechanism can be provided by the variable application of friction to the surface of the first or second support shaft(s) upon rotation of an abutting cam connected to said connector, securing strap or forearm retainer.

In preferred embodiments, said first support shaft(s) and said second support shaft(s) are each formed from carbon fibre, resulting in a stiff but negligible mass support structure that minimises the contribution of these fixed components to the perceived weight and/or dynamic properties of the training device. In some embodiments, the device comprises one first support shaft and one second support shaft. In other embodiments, more than one, e.g. two, three or four, first and/or second support shafts may be provided. Multiple, radially aligned, support shafts may be provided in embodiments where increase device stiffness is required.

In further preferred embodiments, a portion, but not all, of said securing strap is connected to a rigid support layer, wherein said support layer is secured or securable in position on said second support shaft or at least one, and preferably all of, said second support shafts. This arrangement not only facilitates a secure fit around the residual limb of the user but also simplifies the process of fitting the device.

Preferably, the body harness is tethered to said securing strap and/or the proximal part or half of said second support shaft or at least one, and preferably all of, said second support shafts via variable length elastic chord. Whilst not limited to any particular arrangement for achieving such variation of chord length, this can be effectively provided by a cleat configured to securely hold the chord in place and prevent slipping. This arrangement is advantageous in that it allows for adjustment of the position of the securing strap relative to the body harness, thereby optimising the fit around residual limb of a transhumeral amputee user.

The component part referred to as an arm retainer is preferably provided in the form an inflatable armband, which is optionally formed from a thermoplastic sheet material. An exemplary thermoplastic sheet material is a polyurethane (TPU) sheet material, which may be ultrasonically welded to form an airtight enclosure.

Preferably, the arm retainer is disposed within a rigid support structure, e.g. a tube, wherein the support structure is secured or securable in position on said second support shaft or at least one, and preferably all of, said second support shafts. However, as the skilled reader will readily appreciate, the arm retainer may likewise be disposed in a non-rigid inelastic support structure, such as a flexible inelastic strap or mesh. Such a support structure advantageously anchors or secures the arm retainer in position on said support shaft(s), restraining movement of the retainer relative to the support shaft.

As per the first aspect, the connector and/or, if present, said support structure and/or said support layer are preferably formed from a thermoplastic polymer, and more preferably are formed from 3D printable thermoplastic polymer such as acrylonitrile butadiene styrene (ABS), polylactic acid (PLA), polycarbonate (PC), polyamide, high impact polystyrene (HIPS) or high-density polyethylene (HDPE). In particularly preferred embodiments, these components are formed from ABS.

Again as per the first aspect, in preferred embodiments the connector comprises a plurality of radially aligned chambers, wherein each chamber comprises at least one wall and, optionally, a base to define an inner chamber surface that is configured to removably accept a corresponding weight. In a particularly preferred embodiment, the connector comprises 6 chambers, each configured to receive a 75 g weight. Preferably, each inner chamber surface includes or is formed from a magnet, which allows for the secure yet removable attachment of metal weights, thereby allowing the device to replicate the different weights of specific Myo prosthetics.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics or compounds described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Embodiments of the invention will now be described by way of example only with reference to the following figures where:

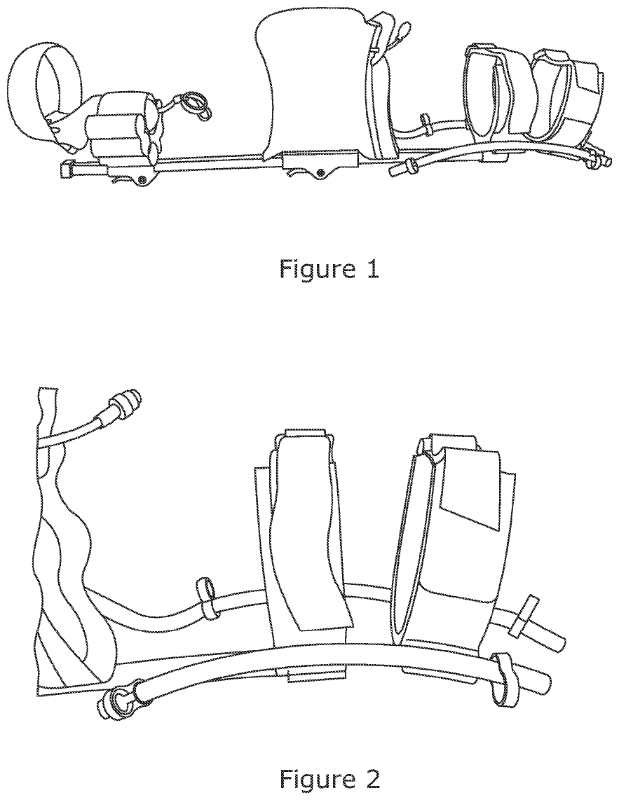

FIG. 1 shows a side view of a prosthetic training device according to the first aspect of the invention;

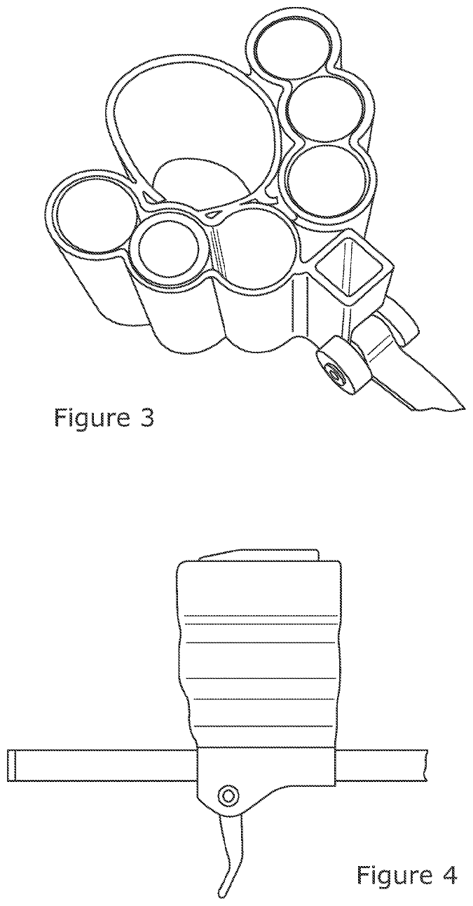

FIG. 2 shows a side view of a section of the training device shown FIG. 1;

FIG. 3 shows a front view of the connector component provided in the training device shown in FIG. 1;

FIG. 4 shows a side view of the connector component shown in FIG. 3;

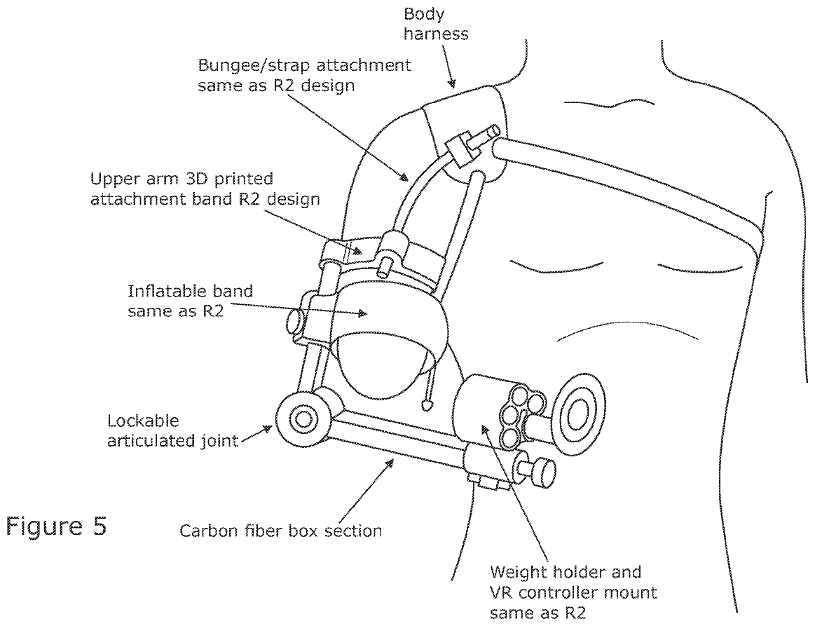

FIG. 5 shows a front representation of a prosthetic training device according to a second aspect of the invention;

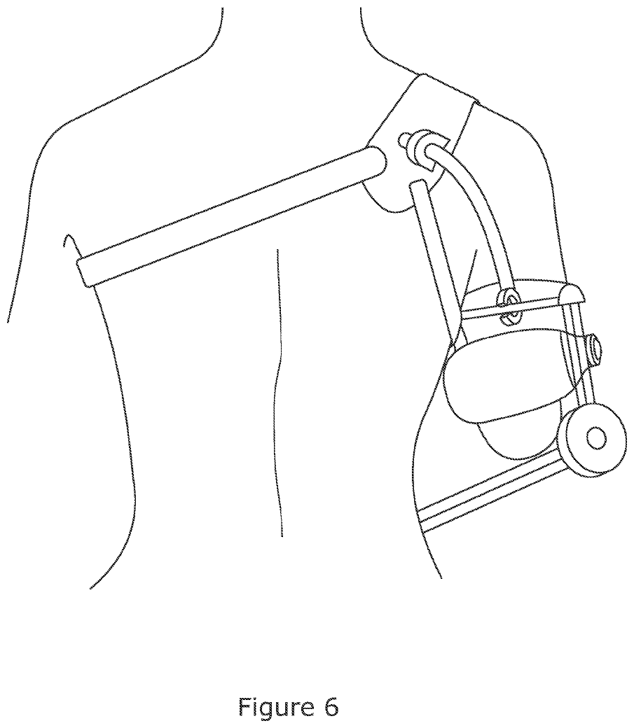

FIG. 6 shows a rear representation of the training device shown in FIG. 5.

Referring firstly to FIG. 1, a side view of a prosthetic training device for attachment to a residual limb of a transradial amputee is shown. An elongate rigid support shaft, formed from carbon fibre box section is provided onto which a number of elements are secured. A connector, comprising six radially aligned chambers each adapted to removably receive a 75 g cylindrical metal weight and a holder adapted to removably receive a VR controller or other positional sensor, is slidably attached to the support shaft and releasably fixed in the desired position in the distal region of the support shaft by a cam lock which modifies the frictional force imparted on the surface of the support shaft by rotation of an abutting cam attached to said connector.

A forearm retainer, provided by way of an inflatable armband, is similarly slidably attached and releasably fixed in position in the central region of the support shaft by a second cam lock. The inflatable armband includes an inflation valve to introduce air into the armband such that, in use, a circumferential force is applied to the residual limb of the user, securing the device to the variable shape and volume of the residual limb of the user whilst avoiding circulation problems within the limb.

Further, a first securing strap is slidably attached and releasably fixed in position in the desired position in the proximal region of the support shaft by a third cam lock. Around 75% of the securing strap is connected to a rigid support layer to which the cam lock mechanism is attached.

A second securing strap is tethered to the first securing strap by way of two elastic chords. Like the first securing strap, around 75% of the securing strap is connected to a rigid support layer.

In use, the VR controller or other or other positional sensor is attached to the connector and an appropriate weight is secured in one or more of radial chambers to mimic the weight of the user's Myo prosthetic device. The connector, armband and first securing strap are then locked into place on the support structure to mimic the dimension of the

Myo prosthetic device. The residual limb of the user is then secured to the device through inflation of the armband, tightening of the first securing strap around the limb below the elbow, and tightening of the second securing strap around the limb above the elbow.

The connector and rigid support layers are all formed from ABS by 3D printing. The inflatable armband is formed from ultrasonically welded TPU.

FIG. 2 shows a close up view of the proximal region of the support shaft and the first and second securing straps, which are tethered together via two lengths of elastic chord, the length of which can be varied by sliding the cleats, which are also formed from ABS by 3D printing, along the length of the chord.

The connector is shown in detail in FIG. 3 and FIG. 4. FIG. 3 shows metal weights (75 g each) are disposed within five of the six radially aligned chambers and secured in place via a magnet disposed in the inner chamber surface. FIG. 4 shows the side profile of the connector attached to the support shaft, with the cam lock in an open position, thereby allowing the connector to slide along the length of the support shaft.

FIG. 5 and FIG. 6 show a similar prosthetic training device, but which has been adapted for attachment to a residual limb of a transhumeral amputee. In such a device, the connector, armband and securing strap (each of which are identical to those used in the device of FIGS. 1 to 4), are secured to a support structure formed from two elongate carbon fibre support shafts which are hingedly connected together via a lockable articulated joint.

A connector, again comprising six radially aligned chambers each adapted to removably receive a 75 g cylindrical metal weight and a holder adapted to removably receive a VR controller or other positional sensor, is slidably attached to the first support shaft and releasably fixed in the desired position by a first cam lock. An arm retainer, provided by way of an inflatable armband, is similarly slidably attached and releasably fixed in position in the central region of the second support shaft by a second cam lock. The inflatable armband again includes an inflation valve to introduce air into the armband such that, in use, a circumferential force is applied to the residual limb of the user, securing the device to the variable shape and volume of the residual limb of the user whilst avoiding circulation problems within the limb.

Further, a securing strap is slidably attached and releasably fixed in the desired position in the proximal region of the second support shaft by a third cam lock. Around 75% of the securing strap is connected to a rigid support layer to which the cam lock mechanism is attached.

As the residual limb lacks an elbow joint, a body harness is used in lieu of a second securing strap, which is tethered to the securing strap by way of two elastic chords. The harness includes a first strap which wraps around the shoulder of the residual limb and a second strap which wraps around the torso of a user.

1. A prosthetic training device for attachment to a residual limb of a transradial amputee, said device comprising:
(a) at least one elongate rigid support shaft;
(b) a connector configured to removably receive one or more weights and/or a virtual reality (VR) controller or other positional sensor, wherein said connector is engaged with and secured or securable in a fixed position along the distal part or half of said support shaft(s);
(c) a first securing strap comprising a first end and a second end, wherein said strap is engaged with and secured or securable in a fixed position along the proximal part or half of at least one of said support shaft(s), and wherein said strap is configured to selectively secure the first end to the second end around the residual limb of a user at a circumferential position below the elbow;
(d) a second securing strap comprising a first end and second end, wherein said strap is flexibly tethered or tetherable to said first securing strap and/or the proximal part or half of at least one of said support shaft(s), and wherein said strap is configured to selectively secure the first end to the second end around the residual limb of a user at a circumferential position above the elbow; and
(e) a forearm retainer configured to maintain a volumetric fit over the residual limb of a user via adjustable circumferential force, wherein said retainer is engaged with and secured or securable in a fixed position on at least one of said support shaft(s) between said connector and said first securing strap.

2. The device according to claim 1, wherein
said connector is slidably engaged with and securable in a plurality of positions along the distal part or half of said support shaft(s);
said first securing strap is slidably engaged with an securable in a plurality of positions along the proximal part or half of said support shaft or at least one of said support shafts; or
said forearm retainer is slidably engaged with an securable in a plurality of positions along the length of said support shaft or at least one of said support shafts.

3. (canceled)

4. (canceled)

5. The device according to claim 2, wherein one or more of said connector, said first securing strap and said forearm retainer is reversibly secured in position on said support shaft(s) by a releasable locking mechanism.

6. The device according to claim 1, wherein said support shaft(s) is/are formed from carbon fibre.

7. The device according to claim 1, wherein a portion of said first and/or second securing strap is connected to a rigid support layer, and wherein said support layer is secured or securable in position on said support shaft or at least one of said support shafts.

8. The device according to claim 1, wherein said forearm retainer is disposed within a rigid support structure or within a non-rigid inelastic support structure, and wherein said support structure is secured or securable in position on said support shaft or at least one of said support shafts.

9. The device according to claim 1, wherein said second securing strap is tethered to said first securing strap and/or the proximal part or half of said support shaft or at least one of said support shafts via variable length elastic chord.

10. A prosthetic training device for attachment to a residual limb of a transhumeral amputee, said device comprising:
(a) at least one first elongate rigid support shaft;
(b) at least one second elongate rigid support shaft, wherein the distal end of each of said at least one second support shaft is hingedly connected to the proximal end of the corresponding first support shaft via an articulated and optionally lockable joint;
(c) a connector configured to removably receive one or more weights and/or a virtual reality (VR) controller or other positional sensor, wherein said connector is engaged with and secured or securable in a fixed position along the distal part or half of said first support shaft(s);
(d) a securing strap comprising a first end and a second end, wherein said strap is engaged with and secured or securable in a fixed position along the proximal part or half of at least one of said second support shaft(s), and wherein said strap is configured to selectively secure the first end to the second end around a circumferential position of the residual limb of a user;
(e) a body harness flexibly tethered or tetherable to said securing strap and/or the proximal part or half of at least one of said second support shaft(s) and configured such that, in use, said harness wraps around the shoulder of the residual limb and around the torso of a user; and
(f) an arm retainer configured to maintain a volumetric fit over a portion of the residual limb of a user via adjustable circumferential force, wherein said retainer is engaged with and secured or securable in a fixed position on at least one of said second support shaft(s) between said articulated joint and said securing strap.

11. The device according to claim 10, wherein said connector is slidably engaged with and securable in a plurality of positions along the length of the first support shaft(s).

12. The device according to claim 10, wherein said securing strap is slidably engaged with and securable in a plurality of positions along the proximal part or half of at least one of said second support shaft(s).

13. The device according to claim 10, wherein said arm retainer is slidably engaged with and securable in a plurality of positions along the length of at least one of said second support shaft(s).

14. The device according to claim 10, wherein one or more of said connector, said securing strap and said arm retainer is reversibly secured in position on said first or said second support shaft(s) by a releasable locking mechanism.

15. The device according to claim 10, wherein said first and said second support shaft(s) are each formed from carbon fibre.

16. The device according to claim 10, wherein a portion of said securing strap is connected to a rigid support layer, and wherein said support layer is secured or securable in position on at least one of said second support shaft(s).

17. The device according to claim 10, wherein said body harness is tethered to said securing strap and/or the proximal part or half of at least one of said second support shaft(s) via variable length elastic chord.

18. The device according to claim 10, wherein said arm retainer is disposed within a rigid support structure or within a non-rigid inelastic support structure, and wherein said support structure is secured or securable in position on at least one of said second support shaft(s).

19. The device according to claim 1, wherein said forearm retainer is an inflatable armband which is optionally formed from a thermoplastic polyurethane (TPU) sheet material.

20. The device according to claim 1, wherein said connector and/or, if present, said support structure and/or said support layer(s) are formed from a thermoplastic polymer, optionally a 3D printable thermoplastic polymer such as acrylonitrile butadiene styrene (ABS), polylactic acid (PLA), polycarbonate (PC), polyamide, high impact polystyrene (HIPS) or high-density polyethylene (HDPE).

21. The device according to claim 1, wherein said connector comprises a plurality of radially aligned chambers, and wherein each chamber comprises at least one wall and, optionally. a base to define an inner chamber surface that is configured to removably accept a corresponding weight.

22. The device according to claim 21, wherein said inner chamber surface includes or is formed from a magnet.
